(19) United States
(12) Patent Application Publication
(10) Pub. No.: US 2003/0059452 A1
(43) Pub. Date: Mar. 27, 2003

(54) Cosmetic composition comprising at least non-polymeric crystalline salt

(76) Inventor: Franck Giroud, Clichy (FR)

Correspondence Address: FINNEGAN, HENDERSON, FARABOW, GARRETT &, DUNNER LLP, 1300 I STREET, NW, WASHINGTON, DC, 20006, US

(21) Appl. No.: 10/239,502

(22) PCT Filed: Mar. 5, 2001
(86) PCT No.: PCT/FR01/00643

(30) Foreign Application Priority Data
Mar. 24, 2000 (FR) 00/03791

(51) Int. Cl. A61K 9/00

(52) U.S. Cl. 424/401

(57) ABSTRACT

The invention concerns a composition comprising at least a non-polymeric crystalline salt and a solvent medium formed by one or several organic solvent(s) and, optionally water: (1) the non-polymeric crystalline salt having a solubility at 20 ° C. in absolute ethanol or in all solvents or mixtures of solvents having Hansen parameters whereof the dispersing constituent δ_{d} is less than 18 Mpa^{0.5}, whereof the polar constituent δₚ is less than 15 Mpa^{0.5} and whereof the hydrogen constituent δₕ is less than 27 Mpa^{0.5}, higher than 20 grams per litre; a solubility in water at 20 ° C. less than 1000 grams per litre; (2) the organic solvent(s) being present in the solvent medium in a proportion higher than 50% relative to the total weight of the solvent medium.

The subject of the invention is a cosmetic composition comprising at least one nonpolymeric crystalline salt and one solvent medium formed by one or more organic solvents, and optionally water. It also relates to the use of such crystalline salts in cosmetic hair compositions, for fixing and/or shaping the hair.

Products for fixing and/or holding the hairstyle are known which exist in the form of lacquers, sprays, gels or lotions. These products contain one or more polymeric materials in solution, which hold the hair in a desired shape. The cohesion of the hair is provided by the formation of physical junctions between the hair strands, consisting of the polymer material or the mixture of polymer materials.

This type of shaping is permanent since any major modifications of the hairstyle cause the destruction of the polymer junctions, in other words the loss of the shape retention of the hair. Conventional hairstyling products do not therefore make it possible to remodel the hairstyle without a fresh supply of product.

For example, when the user of such products combs, and a fortiori washes their hair, after having applied a fixing product, the latter loses its efficacy or disappears. If this user wishes again to fix their hair after washing or combing, they therefore have to apply, after such an operation, a fresh dose of fixing product. This application has the drawback of being time consuming, and of costing every time the price of a new dose of product.

The problem posed by the invention is to provide cosmetic compositions for holding and/or shaping the hair with which it is possible to fix one's hair once, to disarrange it, to restyle it and to fix it again, possibly several times, without needing to apply, after each disarranging, a new dose of hair fixing and/or holding composition, this composition having, in addition, to offer the hair good cosmetic properties such as disentanglement and softness.

To solve this problem, the invention proposes a cosmetic composition comprising at least one nonpolymeric crystalline salt and one solvent medium formed by (i) one or more organic solvents, and optionally (ii) water, characterized in that:

(1) the nonpolymeric crystalline salt has

a solubility at 20° C., in absolute ethanol or in any solvents or solvent mixtures having Hansen solubility parameters whose dispersive component δ_{d} is less than 18 Mpa^{0.5}, whose polar component δₚ is less than 15 Mpa^{0.5} and whose hydrogen component δₕ is less than 27 Mpa^{0.5}, greater than 20 grams per liter;

a solubility, in water at 20° C., less than 1000 grams per liter;

(2) the organic solvent(s) is (are) present in the solvent medium in a proportion greater than 50% relative to the total weight of the solvent medium.

Another subject of the present invention relates to the use of such cosmetic compositions for fixing and/or holding the hairstyle.

Yet another subject of the invention relates to the use of said nonpolymeric crystalline salt as fixing agent in a cosmetic hair composition, and in particular as sole fixing agent.

For the purposes of the present invention, the expression “crystalline salt” is understood to mean any salt possessing an organization giving rise to a crystallographic network, for example, of the triclinic, monoclinic, orthorhombic, hexagonal, rhombohedral, tetragonal or cubic type.

The salt may be organic or inorganic.

The inorganic bases, which may be used in hair cosmetics as agent for neutralizing fixing polymers, do not form part of the crystalline salts entering into the constitution of the compositions according to the present invention. For example, potassium hydroxide (KOH) or sodium hydroxide (NaOH), which may be used as neutralizing agent in lacquers, do not form part of the crystalline salts targeted in this patent application.

Advantageously, the nonpolymeric crystalline salt is present in the composition in accordance with the invention at a minimum concentration equal to 0.5% by weight relative to the total weight of the composition.

The expression “solubility of a compound in a liquid medium” is understood to mean the quantity of this compound which can dissolve in one liter of this liquid.

In accordance with the invention, the nonpolymeric crystalline salt should be soluble at 20° C. at a concentration greater than 20 g/l in absolute ethanol or in any solvents or solvent mixtures having Hansen solubility parameters whose dispersive component δ_{d} is less than 18 Mpa^{0.5}, whose polar component δₚ is less than 15 Mpa^{0.5} and whose hydrogen component δₕ is less than 27 Mpa^{0.5}. For the definition and the values of the components of the Hansen solubility parameters, reference should be made to “Handbook of Solubility Parameters and Other Cohesion Parameters” Chapters 5.9, 7.1 and 8.3 Allan F. M. Barton CRC Press ISBN 0-8493-3295-8.

The solubility measurements are carried out at normal atmospheric pressure.

Preferably, the nonpolymeric crystalline salt has a solubility, in absolute ethanol at 20° C., greater than 20 grams per liter, and preferably greater than 100 grams per liter. Moreover, the nonpolymeric crystalline salt preferably has a solubility, in water at 20° C., less than 1000 grams per liter, and preferably less than 100 grams per liter.

Advantageously, the organic solvent(s) is (are) present in the solvent medium in a proportion greater than 80% relative to the total weight of the solvent medium and are chosen from the group comprising C₁ to C₄ alcohols, such as ethanol or isopropanol, C₆ to C₁₀ alkanes, acetone, methyl ethyl ketone, methyl acetate, butyl acetate, ethyl acetate, dimethoxyethane, diethoxyethane and mixtures thereof, ethanol being preferred.

“The hairstyling effect” represents the capacity of the composition to fix and/or hold the hairstyle after application of a dose of product. “The hair restyling effect” represents the same capacity, when the user has disarranged and then restyled their hair at least once after the application of the dose of product, without applying a fresh dose.

To obtain an optimum hairstyling and restyling effect, the preferred nonpolymeric crystalline salt being calcium salicylate.

In accordance with the invention, the composition advantageously contains, in addition, conventional cosmetic additives chosen from thickeners, anionic, nonionic, cationic or amphoteric surfactants, perfumes, preservatives, sunscreens, proteins, vitamins, provitamins, anionic, nonionic, cationic or amphoteric nonfixing polymers, mineral, vegetable or synthetic oils, ceramides, pseudoceramides, volatile or nonvolatile, linear or cyclic, modified or unmodified silicones and any other additive conventionally used in cosmetic compositions intended to be applied to the hair.

When the composition according to the invention is packaged in an aerosol device, it comprises at least one propellant which may be chosen from volatile hydrocarbons such as n-butane, propane, isobutane, pentane, halogenated hydrocarbons and mixtures thereof. It is also possible to use, as propellant, carbon dioxide gas, nitrous oxide, dimethyl ether (DME), nitrogen, compressed air. It is also possible to use mixtures of propellants. Preferably, dimethyl ether is used.

Advantageously, the propellant is present at a concentration of between 5 and 90% by weight relative to the total weight of the composition in the aerosol device, and more particularly at a concentration of between 10 and 60%.

The compositions in accordance with the invention may be applied to dry or wet hair.

The invention will be more fully illustrated using the following nonlimiting example.

All the percentages are relative percentages by weight relative to the total weight of the composition and a.s. means active substance.

### EXAMPLE

5 g of composition 1 according to the invention described below are applied to a head having chestnut brown European hair about 20 cm long, by spraying using a spray.

5 g of hairstyling gel consisting of a composition 2 according to the prior art are applied to another head of the same type. This gel is used as control hairstyling product for the demonstration of the effect.

Composition 1 (invention):

| | |
|---|---|
| Preservative | 0.1% |
| Perfume | 0.2% |
| Calcium salicylate: | 2.5% |
| Water: | 10% |
| Absolute ethanol: | 87.2% |

Composition 2 (prior art):

| | |
|---|---|
| Preservative | 0.1% |
| Perfume | 0.2% |
| Synthalen K(1) | 1.5% |
| Absolute ethanol | 48.6% |
| Water | 49.6% |

(1)Polyacrylic acid marketed by the company 3V

Synthalen K is a known fixing polymer used in hair fixing compositions.

To visualize the hairstyling restyling effect, the hair is first of all disentangled using a comb or a brush. Once this operation has been performed, the hair is combed from back to front, so as to bring the hair to the front of the face.

With the formula of the invention it is noted that the hair remain fixed in the position imposed by the combing, and although this position is not that of the natural implantation of the hair.

With the control hairstyling gel, it is impossible to bring the hair to the front of the face. The hair is standing on end, the hairstyle is soft and difficult to reshape.

It is also noted that the formula of the invention makes it possible to modify the hairstyle repeatedly, without a fresh supply of product. For the hairstyling gel, reshaping of the hair is only possible providing the hair is wetted or a fresh application of gel is made.

From a cosmetic point of view, the formula of the invention gives a noncharged and nonsticky feel unlike that obtained after application of the control gel.

Furthermore, the formula of the invention leaves hair separated from each other, unlike the gel which gives rise to the formation of hair locks stuck to each other, the hair therefore appearing visually more natural.

1. A cosmetic composition comprising at least one nonpolymeric crystalline salt and one solvent medium formed by (i) one or more organic solvents, and optionally (ii) water, characterized in that:
(1) the nonpolymeric crystalline salt has
a solubility at 20° C., in absolute ethanol or in any solvents or solvent mixtures having Hansen solubility parameters whose dispersive component δ_{d} is less than 18 Mpa^{0.5}, whose polar component δₚ is less than 15 Mpa^{0.5} and whose hydrogen component δₕ is less than 27 Mpa^{0.5}, greater than 20 grams per liter;
a solubility, in water at 20° C., less than 1000 grams per liter;
(2) the organic solvent(s) is (are) present in the solvent medium in a proportion greater than 50% relative to the total weight of the solvent medium.

2. The cosmetic composition as claimed in claim 1, characterized in that the nonpolymeric crystalline salt is present in the composition at a minimum concentration equal to 0.5% by weight relative to the total weight of the composition.

3. The cosmetic composition as claimed in any one of the preceding claims, characterized in that the nonpolymeric crystalline salt has a solubility, in absolute ethanol at 20° C., greater than 20 grams per liter, and preferably greater than 100 grams per liter.

4. The cosmetic composition as claimed in any one of the preceding claims, characterized in that the nonpolymeric crystalline salt has a solubility, in water at 20° C., less than 1000 grams per liter, and preferably less than 100 grams per liter.

5. The composition as claimed in any one of the preceding claims, characterized in that the organic solvent(s) is (are) present in the solvent medium in a proportion greater than 80% relative to the total weight of the solvent medium.

6. The composition as claimed in any one of the preceding claims, characterized in that the organic solvents are chosen from the group comprising C₁ to C₄ alcohols, such as ethanol or isopropanol, C₆ to C₁₀ alkanes, acetone, methyl ethyl ketone, methyl acetate, butyl acetate, ethyl acetate, dimethoxyethane, diethoxyethane and mixtures thereof.

7. The composition as claimed in any one of the preceding claims, characterized in that the organic solvent consists of absolute ethanol.

8. A composition, characterized in that the nonpolymeric crystalline salt used is calcium salicylate.

9. The composition as claimed in any one of the preceding claims, characterized in that it contains, in addition, conventional cosmetic additives chosen from thickeners, anionic, nonionic, cationic or amphoteric surfactants, perfumes, preservatives, sunscreens, proteins, vitamins, provitamins, anionic, nonionic, cationic or amphoteric nonfixing polymers, mineral, vegetable or synthetic oils, ceramides, pseudoceramides, volatile or nonvolatile, linear or cyclic, modified or unmodified silicones and any other additive conventionally used in cosmetic compositions intended to be applied to the hair.

10. An aerosol device comprising a composition according to any one of the preceding claims and at least one propellant chosen from volatile hydrocarbons such as n-butane, propane, isobutane, pentane, halogenated hydrocarbons, carbon dioxide gas, nitrous oxide, dimethyl ether (DME), nitrogen, compressed air or a mixture of these propellants.

11. The use of a composition as claimed in any one of the preceding claims for fixing and/or holding the hairstyle.

12. The use of a nonpolymeric crystalline salt comprising:
a solubility at 20° C., in absolute ethanol or in any solvents or solvent mixtures having Hansen solubility parameters whose dispersive component δ_{d} is less than 18 Mpa^{0.5}, whose polar component δₚ is less than 15 Mpa^{0.5} and whose hydrogen component δₕ is less than 27 Mpa^{0.5}, greater than 20 grams per liter;
a solubility, in water at 20° C., less than 1000 grams per liter;
as fixing agent in a cosmetic hair composition.

13. The use of a crystalline salt as claimed in claim 12, as sole fixing agent in a hair composition.
